SEQUENCE LISTING (12 sequences)

SEQ ID NO: 1 - DNA, 5387 bp, Merkel cell Polyomavirus MCV-IC13
ggggctcctagcctccgaggcctctggaaaaaaaagagagaggcctctgaggcttaagaggcttaattagcaaaaaaggcagtatctaagggcagatcccaagggcgggaaactgcagtataaaaaccactccttagtgaggtagctcatttgctcctctgctctttctgcaaactccttctgcatatagacaagatggatttagtcctaaataggaaagaaagagaggctctctgcaagcttttagagattgctcctagttgttatggcaacatccctctgatgaaagctgctttcaaaagaagctgcttaaagcatcaccctgataaagggggaaatcctgttataatgatggaattgaacaccctttggagcaaattccagcaaaatatccacaagctcagaagtgacttctctatgtttgatgaggtcagtacaaaatttccttgggaagaatatggaactttaaaggattatatgcaaagtggatataatgctagattttgcagaggtcctgggtgcatgcttaagcaacttagagattctaagtgcgcttgtattagctgtaagttgtctcgccagcattgtagtctaaaaactttaaagcaaaaaaactgtctgacgtggggagagtgtttttgctatcagtgctttattctttggtttggatttcctcctacttgggaaagttttgactggtggcaaaaaactttagaagaaactgactactgcttactgcatctgcaccttttctagactcctacttccttcctctgtaagtattagatatggaaaagtctataaggcaaaatatcaaagaaaggttatttatgacagattttctgtactttcccatctaggttgacgaggcccctatatatgggaccactaaattcaaagaatggtggagatcaggaggattcagcttcgggaaggcatacgaatatgggcccaatccacacgggaccaactcaagatccagaaagccttcctccaatgcatccaggggagcccccagtggaagctcaccaccccacagccagagctcttcctctgggtatgggtccttctcagcgtcccaggcttcagactcccagtccagaggacccgatatacctcccgaacaccatgaggaacccacctcatcctctggatccagtagcagaggggagaccaccaattcaggaagagaatccagcacacccaatggaaccagtgtacctagaaattcttccagaacggatggcacctgggaggatctcttctgcgatgaatcactttcctcccctgagcctccctcgtcctctgaggagcctgaggagcccccctcctcaagaagctcgccccggcagcccccgtcttcctctgccgaggaggcctcatcatctcagtttacagatgaggaatacagatcctcctccttcaccaccccgaagacccctcctccattctcaagaaagcgaaaatttggggggtcccgaagctctgcaagctctgctagttcagcaagttttacaagcactccaccaaagccaaaaaagaacagagaaactcctgttcctactgattttcctattgatctttctgattatcttagccatgctgtatatagtaataaaacagtaagttgttttgccatttatactacttctgataaagctatagagttatatgataagattgagaaatttaaagttgattttaaaagcaggcatgcctgtgaattaggatgtattttattgtttataactttatcaaagcatagagtatctgctattaagaatttttgctctaccttctgcactataagctttttaatttgtaaaggagtgaataagatgcctgaaatgtataataatttatgcaagcccccttacaaattactgcaagagaataagccactgctcaattatgaatttcaagaaaaagaaaaagaggccagctgtaattggaatttagttgctgaatttgcttgtgaatatgagctagacgaccactttattatcttagcccattatctagactttgcaaaaccatttccttgccaaaagtgtgaaaatagatctcgcctcaaacctcacaaggctcatgaggctcatcattctaatgctaagctattttatgaatctaaatctcagaaaaccatttgccaacaagccgcagacactgttctagccaaaaggaggttagagatgctggaaatgaccaggacagaaatgctatgtaagaagtttaagaagcacctagagagattaagagatttagatacaatagatctactgtattatatgggtggtgtggcctggtactgctgcttatttgaagagtttgaaaagaagctgcagaaaattattcaattattaacagagaatatacctaagtatagaaacatttggtttaaagggcctattaacagtggaaaaacaagctttgctgcagccttaatagatttgctagaagggaaggccttgaatataaactgtccatctgataaactgccttttgaactaggatgtgctttggataaatttatggttgtttttgaggatgtgaaagggcaaaatagcctaaataaagatctgcaaccagggcaaggaataaataaccttgataacttaagagatcatctagatggtgctgtagctgtaagcttagagaagaagcatgtgaataaaaagcatcagatttttcctccttgtattgttactgctaatgattattttattcccaaaacattaatagcaagatttagttatactttacacttttccccaaaggcaaatctaagagattccctggatcagaacatggaaataagaaaaagaagaattcttcaaagtggaaccactttattgctttgtcttatttggtgcttgcctgatacaacctttaagccttgcttacaagaagaaattaaaaactggaagcaaattttacagagtgaaatatcatatggtaaattttgtcaaatgatagaaaatgtagaagctggtcaggaccctctgctcaatattcttattgaggaagagggccctgaggaaactgaagaaacccaagattctggtactttttctcaataaaggcatctgcttcatatttcctgtgtttgtttttctggggcctacttaactgaataggaatgcatgaaataattctcataattcttgtgtttggctttctttttgagaggccttttgaggtcctttcagtggcgccttgcccttatcctgctgattactttggaatgttactgctgctggggcaacagagggctttgggtaaacagttttctcctgcccaaatttatctaaaaatctgacaatatcaggatcaccaggtaattgttctgacccctcatatattctaacctcttctacctgattatcttttccttccataggttggcctgacacttttggcattaagttgctgaagagtgagtttattaaattaactactgggtaggggtttttcacccatctttttctgaaagtaacattaaaatatctaggcaacccatgaagagccatttttccactggttttaaacagaaaccccactatgtctgcacagctaataaataggccatctcctttgcatagagggcccactccattctcatctaaaaggacagtagttagagtattactaaattgaagaactgtaggagtctgagagcctgtctgaatagacccatagtatctactgttttcatttttagaaggatcaggacaccatacttctataggataatttccatctttatctaattttgctttagcttgtggatctaggccctgatttttaggtgtcatttttcttcccaatacagtttcaattgtaataggcccaccatttgtagtttttggatactcagtctggtaatctaaaactaggccttgcaaatccagaggttctcccccaatggcaaacatatggtaatttactcctgacacaggaataccagcaccataatcatgaactcttttcatgtcccaataatgaacattaattaaagaacttattccaactacttctgttttaacagatattgcctcccacatctgcaatgtgtcacaggtaatatcctcatttagcattggcagagacactcttgccacactgtaagctggcaaattttccttgatgggctgatctggagatgatccctttggctgcaggtcataagtataagtataccagtttgaagtagtaggaagatcaggggaattaactcccattcttggattcaaatacaactcaatttgggtaatgctatcttctccagtaaccacagttaatacttccactcctcctttaacaagcagttttggaactgaggcaacattagggcagcatcccggcttaggtatacattgccttttgggtgttttacaggtggatgatgcttttctttttggtgccatcttcaattacttgtaattcaggagaaatatatccactaaggcctagtaccagaggaagaagccaatctggagtttgctgctgcagagttcctcctatatgttcaggaattaatatagcctctcctgataaaaggccctgattctgagaagcagttgtgtgaaagacccaccggctatttagtatcagattcactaggtttgattgtatctgcagcctagaggtaggagataaagaattaaaaatattttgccccacagaatgcagcaagctattttcccactgcagaggatctaggctaaaggccataagtgcatgcctcaaaacctcattactacctacccacgaaacatccctctttacaagtgacacttgctcgcgtgacaacctcacccccacagttattagagagcctataccactaacagtttggagaatgaagccataagttaaaccttggttaaccaaagaagccactaatgagaaatttgaaaactgttcagctgtgaacccaagttgagctaaagcctcaatgccagaaataccctcaattgtcattaaactggagatctctgcttccaaagctgctaaagcttctcctgtaagaatagcttccaaagttactcctgtggtggcacttagttcagtagcaatttcaccaatattggccagcagtgtgatgatgccccccatcctgaaaaataaataaggatacttactcttttaatgtcctcctccctttgtaagagaaaaaaaagcctccgggcctcccttgttgaaaaaaagttaagagtcttccgtctccctcccaaacagaaagaaaaaaagttttgtttatcagtcaaactccgcctctccaggaaatgagtcaatgccagaaaccctgcagcaataaaagttcaatcatgtaaccacaacttggctgcctaggtgacttttttttttcaagttggcagaggctt

SEQ ID NO: 2 - DNA, 561 bp, Merkel cell Polyomavirus MCV-IC13
atggatttagtcctaaataggaaagaaagagaggctctctgcaagcttttagagattgctcctagttgttatggcaacatccctctgatgaaagctgctttcaaaagaagctgcttaaagcatcaccctgataaagggggaaatcctgttataatgatggaattgaacaccctttggagcaaattccagcaaaatatccacaagctcagaagtgacttctctatgtttgatgaggtcagtacaaaatttccttgggaagaatatggaactttaaaggattatatgcaaagtggatataatgctagattttgcagaggtcctgggtgcatgcttaagcaacttagagattctaagtgcgcttgtattagctgtaagttgtctcgccagcattgtagtctaaaaactttaaagcaaaaaaactgtctgacgtggggagagtgtttttgctatcagtgctttattctttggtttggatttcctcctacttgggaaagttttgactggtggcaaaaaactttagaagaaactgactactgcttactgcatctgcaccttttctag

SEQ ID NO: 3 - protein, 186 aa, Merkel cell Polyomavirus MCV-IC13
MDLVLNRKEREALCKLLEIAPSCYGNIPLMKAAFKRSCLKHHPDKGGNPVIMMELNTLWSKFQQNIHKLRSDFSMFDEVSTKFPWEEYGTLKDYMQSGYNARFCRGPGCMLKQLRDSKCACISCKLSRQHCSLKTLKQKNCLTWGECFCYQCFILWFGFPPTWESFDWWQKTLEETDYCLLHLHLF

SEQ ID NO: 4 - DNA, 234 bp, Merkel cell Polyomavirus MCV-IC13
atggatttagtcctaaataggaaagaaagagaggctctctgcaagcttttagagattgctcctagttgttatggcaacatccctctgatgaaagctgctttcaaaagaagctgcttaaagcatcaccctgataaagggggaaatcctgttataatgatggaattgaacaccctttggagcaaattccagcaaaatatccacaagctcagaagtgacttctctatgtttgatgag

SEQ ID NO: 5 - DNA, 2220 bp, Merkel cell Polyomavirus MCV-IC13
gttgacgaggcccctatatatgggaccactaaattcaaagaatggtggagatcaggaggattcagcttcgggaaggcatacgaatatgggcccaatccacacgggaccaactcaagatccagaaagccttcctccaatgcatccaggggagcccccagtggaagctcaccaccccacagccagagctcttcctctgggtatgggtccttctcagcgtcccaggcttcagactcccagtccagaggacccgatatacctcccgaacaccatgaggaacccacctcatcctctggatccagtagcagaggggagaccaccaattcaggaagagaatccagcacacccaatggaaccagtgtacctagaaattcttccagaacggatggcacctgggaggatctcttctgcgatgaatcactttcctcccctgagcctccctcgtcctctgaggagcctgaggagcccccctcctcaagaagctcgccccggcagcccccgtcttcctctgccgaggaggcctcatcatctcagtttacagatgaggaatacagatcctcctccttcaccaccccgaagacccctcctccattctcaagaaagcgaaaatttggggggtcccgaagctctgcaagctctgctagttcagcaagttttacaagcactccaccaaagccaaaaaagaacagagaaactcctgttcctactgattttcctattgatctttctgattatcttagccatgctgtatatagtaataaaacagtaagttgttttgccatttatactacttctgataaagctatagagttatatgataagattgagaaatttaaagttgattttaaaagcaggcatgcctgtgaattaggatgtattttattgtttataactttatcaaagcatagagtatctgctattaagaatttttgctctaccttctgcactataagctttttaatttgtaaaggagtgaataagatgcctgaaatgtataataatttatgcaagcccccttacaaattactgcaagagaataagccactgctcaattatgaatttcaagaaaaagaaaaagaggccagctgtaattggaatttagttgctgaatttgcttgtgaatatgagctagacgaccactttattatcttagcccattatctagactttgcaaaaccatttccttgccaaaagtgtgaaaatagatctcgcctcaaacctcacaaggctcatgaggctcatcattctaatgctaagctattttatgaatctaaatctcagaaaaccatttgccaacaagccgcagacactgttctagccaaaaggaggttagagatgctggaaatgaccaggacagaaatgctatgtaagaagtttaagaagcacctagagagattaagagatttagatacaatagatctactgtattatatgggtggtgtggcctggtactgctgcttatttgaagagtttgaaaagaagctgcagaaaattattcaattattaacagagaatatacctaagtatagaaacatttggtttaaagggcctattaacagtggaaaaacaagctttgctgcagccttaatagatttgctagaagggaaggccttgaatataaactgtccatctgataaactgccttttgaactaggatgtgctttggataaatttatggttgtttttgaggatgtgaaagggcaaaatagcctaaataaagatctgcaaccagggcaaggaataaataaccttgataacttaagagatcatctagatggtgctgtagctgtaagcttagagaagaagcatgtgaataaaaagcatcagatttttcctccttgtattgttactgctaatgattattttattcccaaaacattaatagcaagatttagttatactttacacttttccccaaaggcaaatctaagagattccctggatcagaacatggaaataagaaaaagaagaattcttcaaagtggaaccactttattgctttgtcttatttggtgcttgcctgatacaacctttaagccttgcttacaagaagaaattaaaaactggaagcaaattttacagagtgaaatatcatatggtaaattttgtcaaatgatagaaaatgtagaagctggtcaggaccctctgctcaatattcttattgaggaagagggccctgaggaaactgaagaaacccaagattctggtactttttctcaataa

SEQ ID NO: 6 - protein, 817 aa, Merkel cell Polyomavirus MCV-IC13
MDLVLNRKEREALCKLLEIAPSCYGNIPLMKAAFKRSCLKHHPDKGGNPVIMMELNTLWSKFQQNIHKLRSDFSMFDEVDEAPIYGTTKFKEWWRSGGFSFGKAYEYGPNPHGTNSRSRKPSSNASRGAPSGSSPPHSQSSSSGYGSFSASQASDSQSRGPDIPPEHHEEPTSSSGSSSRGETTNSGRESSTPNGTSVPRNSSRTDGTWEDLFCDESLSSPEPPSSSEEPEEPPSSRSSPRQPPSSSAEEASSSQFTDEEYRSSSFTTPKTPPPFSRKRKFGGSRSSASSASSASFTSTPPKPKKNRETPVPTDFPIDLSDYLSHAVYSNKTVSCFAIYTTSDKAIELYDKIEKFKVDFKSRHACELGCILLFITLSKHRVSAIKNFCSTFCTISFLICKGVNKMPEMYNNLCKPPYKLLQENKPLLNYEFQEKEKEASCNWNLVAEFACEYELDDHFIILAHYLDFAKPFPCQKCENRSRLKPHKAHEAHHSNAKLFYESKSQKTICQQAADTVLAKRRLEMLEMTRTEMLCKKFKKHLERLRDLDTIDLLYYMGGVAWYCCLFEEFEKKLQKIIQLLTENIPKYRNIWFKGPINSGKTSFAAALIDLLEGKALNINCPSDKLPFELGCALDKFMVVFEDVKGQNSLNKDLQPGQGINNLDNLRDHLDGAVAVSLEKKHVNKKHQIFPPCIVTANDYFIPKTLIARFSYTLHFSPKANLRDSLDQNMEIRKRRILQSGTTLLLCLIWCLPDTTFKPCLQEEIKNWKQILQSEISYGKFCQMIENVEAGQDPLLNILIEEEGPEETEETQDSGTFSQ

SEQ ID NO: 7 - DNA, 1269 bp, Merkel cell Polyomavirus MCV-IC13
tcataattcttgtgtttggctttctttttgagaggccttttgaggtcctttcagtggcgccttgcccttatcctgctgattactttggaatgttactgctgctggggcaacagagggctttgggtaaacagttttctcctgcccaaatttatctaaaaatctgacaatatcaggatcaccaggtaattgttctgacccctcatatattctaacctcttctacctgattatcttttccttccataggttggcctgacacttttggcattaagttgctgaagagtgagtttattaaattaactactgggtaggggtttttcacccatctttttctgaaagtaacattaaaatatctaggcaacccatgaagagccatttttccactggttttaaacagaaaccccactatgtctgcacagctaataaataggccatctcctttgcatagagggcccactccattctcatctaaaaggacagtagttagagtattactaaattgaagaactgtaggagtctgagagcctgtctgaatagacccatagtatctactgttttcatttttagaaggatcaggacaccatacttctataggataatttccatctttatctaattttgctttagcttgtggatctaggccctgatttttaggtgtcatttttcttcccaatacagtttcaattgtaataggcccaccatttgtagtttttggatactcagtctggtaatctaaaactaggccttgcaaatccagaggttctcccccaatggcaaacatatggtaatttactcctgacacaggaataccagcaccataatcatgaactcttttcatgtcccaataatgaacattaattaaagaacttattccaactacttctgttttaacagatattgcctcccacatctgcaatgtgtcacaggtaatatcctcatttagcattggcagagacactcttgccacactgtaagctggcaaattttccttgatgggctgatctggagatgatccctttggctgcaggtcataagtataagtataccagtttgaagtagtaggaagatcaggggaattaactcccattcttggattcaaatacaactcaatttgggtaatgctatcttctccagtaaccacagttaatacttccactcctcctttaacaagcagttttggaactgaggcaacattagggcagcatcccggcttaggtatacattgccttttgggtgttttacaggtggatgatgcttttctttttggtgc

SEQ ID NO: 8 - protein, 423 aa, Merkel cell Polyomavirus MCV-IC13
MAPKRKASSTCKTPKRQCIPKPGCCPNVASVPKLLVKGGVEVLTVVTGEDSITQIELYLNPRMGVNSPDLPTTSNWYTYTYDLQPKGSSPDQPIKENLPAYSVARVSLPMLNEDITCDTLQMWEAISVKTEVVGISSLINVHYWDMKRVHDYGAGIPVSGVNYHMFAIGGEPLDLQGLVLDYQTEYPKTTNGGPITIETVLGRKMTPKNQGLDPQAKAKLDKDGNYPIEVWCPDPSKNENSRYYGSIQTGSQTPTVLQFSNTLTTVLLDENGVGPLCKGDGLFISCADIVGFLFKTSGKMALHGLPRYFNVTFRKRWVKNPYPVVNLINSLFSNLMPKVSGQPMEGKDNQVEEVRIYEGSEQLPGDPDIVRFLDKFGQEKTVYPKPSVAPAAVTFQSNQQDKGKAPLKGPQKASQKESQTQEL

SEQ ID NO: 9 - DNA, 725 bp, Merkel cell Polyomavirus MCV-IC13
tacaggtggatgatgcttttctttttggtgccatcttcaattacttgtaattcaggagaaatatatccactaaggcctagtaccagaggaagaagccaatctggagtttgctgctgcagagttcctcctatatgttcaggaattaatatagcctctcctgataaaaggccctgattctgagaagcagttgtgtgaaagacccaccggctatttagtatcagattcactaggtttgattgtatctgcagcctagaggtaggagataaagaattaaaaatattttgccccacagaatgcagcaagctattttcccactgcagaggatctaggctaaaggccataagtgcatgcctcaaaacctcattactacctacccacgaaacatccctctttacaagtgacacttgctcgcgtgacaacctcacccccacagttattagagagcctataccactaacagtttggagaatgaagccataagttaaaccttggttaaccaaagaagccactaatgagaaatttgaaaactgttcagctgtgaacccaagttgagctaaagcctcaatgccagaaataccctcaattgtcattaaactggagatctctgcttccaaagctgctaaagcttctcctgtaagaatagcttccaaagttactcctgtggtggcacttagttcagtagcaatttcaccaatattggccagcagtgtgatgatgccccccat

SEQ ID NO: 10 - protein, 240 aa, Merkel cell Polyomavirus MCV-IC13
MGGIITLLANIGEIATELSATTGVTLEAILTGEALAALEAEISSLMTIEGISGIEALAQLGFTAEQFSNFSLVASLVNQGLTYGFILQTVSGIGSLITVGVRLSREQVSLVKRDVSWVGSNEVLRHALMAFSLDPLQWENSLLHSVGQNIFNSLSPTSRLQIQSNLVNLILNSRWVFHTTASQNQGLLSGEAILIPEHIGGTLQQQTPDWLLPLVLGLSGYISPELQVIEDGTKKKSIIH

SEQ ID NO: 11 - DNA, 591 bp, Merkel cell Polyomavirus MCV-IC13
ttacaggtggatgatgcttttctttttggtgccatcttcaattacttgtaattcaggagaaatatatccactaaggcctagtaccagaggaagaagccaatctggagtttgctgctgcagagttcctcctatatgttcaggaattaatatagcctctcctgataaaaggccctgattctgagaagcagttgtgtgaaagacccaccggctatttagtatcagattcactaggtttgattgtatctgcagcctagaggtaggagataaagaattaaaaatattttgccccacagaatgcagcaagctattttcccactgcagaggatctaggctaaaggccataagtgcatgcctcaaaacctcattactacctacccacgaaacatccctctttacaagtgacacttgctcgcgtgacaacctcacccccacagttattagagagcctataccactaacagtttggagaatgaagccataagttaaaccttggttaaccaaagaagccactaatgagaaatttgaaaactgttcagctgtgaacccaagttgagctaaagcctcaatgccagaaataccctcaattgtcat

SEQ ID NO: 12 - protein, 181 aa, Merkel cell Polyomavirus MCV-IC13
MTIEGISGIEALAQLGFTAEQFSNFSLVASLVNQGLTYGFILQTVSGIGSLITVGVRLSREQVSLVKRDVSWVGSNEVLRHALMAFSLDPLQWENSLLHSVGQNIFNSLSPTSRLQIQSNLVNLILNSRWVFHTTASQNQGLLSGEAILIPEHIGGTLQQQTPDWLLPLVLGLSGYISPEL

(19) United States
(12) Patent Application Publication
(10) Pub. No.: US 2011/0182901 A1
(43) Pub. Date: Jul. 28, 2011

(54) FULL LENGTH LARGE T TUMOR ANTIGEN OF MERKEL CELL POLYOMAVIRUS AS A THERAPEUTIC TARGET IN MERKEL CELL CARCINOMA

(75) Inventors: Martine PETER, Paris (FR); Xavier SASTRE-GARAU, Paris (FR)

(73) Assignee: INSTITUT CURIE, Paris Cedex 5 (FR)

(21) Appl. No.: 13/004,158

(22) Filed: Jan. 11, 2011

Related U.S. Application Data
(60) Provisional application No. 61/298,343, filed on Jan. 26, 2010.

(51) Int. Cl. A61K 39/395; C12N 7/00; C07H 21/00; C07K 14/01; C07K 16/08; C12Q 1/70; G01N 33/68; G01N 33/53; A61K 31/7088; A61K 39/12; A61P 31/20; A61P 35/00; A61P 37/04

(52) U.S. Cl. 424/139.1; 435/235.1; 536/23.72; 536/23.1; 530/350; 530/389.4; 530/387.9; 435/5; 436/501; 436/86; 436/94; 514/44 R; 424/186.1; 424/204.1

(57) ABSTRACT

The invention relates to a full length Large T tumor antigen of Merkel Cell Polyomavirus (MCV) as a therapeutic target in Merkel Cell Carcinoma (MCC).

### FIELD OF THE INVENTION

The invention relates to a full length Large T tumor antigen of Merkel Cell Polyomavirus as a therapeutic target in Merkel Cell Carcinoma.

### BACKGROUND OF THE INVENTION

Merkel cell carcinoma (MCC) is a cutaneous tumour [1] with neuroendocrine features and poor outcome [2-4]. This tumour develops in the sun-exposed areas of the skin in elderly or immunosuppressed individuals [5]. A three-fold increase in incidence has been observed in the United States over the past 15 years [6], attributable in part to an aging population with extensive sun exposure. Although classically believed to be derived from Merkel neuroendocrine epidermal cells [2], the histogenesis of this tumour is still under debate [3]. Studies on the molecular origins of MCC thus far have provided only negative results [7] and this lack of knowledge limits the development of targeted therapies.

Polyomaviruses are small non enveloped viruses with a double stranded circular DNA chromosome composed of 4700 to 5400 bp. This genome encodes the three structural proteins which constitute the viral particle (VP1, 2, 3) and two early tumour antigens, called small T (ST) and large T (LT). In their natural host, polyomaviruses are non-oncogenic: infection leads to the replication of the viral genome and production of viral particles resulting in cell lysis. In contrast, in heterologous experimentally transformed cells, no production of infectious virus occurs. The non permissive host cells integrate into their genome viral DNA sequences which constitutively express ST and LT antigens.

A new type of human polyomavirus was recently identified in MCC, thus called Merkel Cell Polyomavirus (MCV or MCPyV) [8]. The presence of MCV DNA sequences in MCC has been confirmed in three series of cases [9-11]. This association does not however establish a causal role for MCV in MCC.

In particular, these authors have identified and sequenced two clones of MCV: MCV350 and MCV339. The complete sequences of MCV350 and MCV339 are available under Genbank accession numbers EU375803 and EU375804, respectively.

Document WO 2009/079481 describes isolated or substantially purified polypeptides, nucleic acids, and virus-like particles (VLPs) derived from said clones. A common feature of MCV350 and MCV339 and other clones of MCV found in association with cancerous tissues is that the sequence encoding the Large T antigen (LT) results in a truncated protein. The authors speculate that tumours strongly select against retention of intact MCV large antigen.

Surprisingly, the inventors have identified a novel clone of MCV associated with MCC, named MCV IC-13, which contains a full length LT antigen.

### SUMMARY OF THE INVENTION

The inventors have identified a novel clone of Merkel Cell Polyomavirus (MCV) associated with MCC, whose genome presents several differences compared to the known clones of MCV and in particular which encodes a full length LT antigen.

Thus, in one aspect, the invention relates to a virus-like particle (VLP) wherein said VLP is derived from a polyomavirus, preferably a Merkel Cell Polyomavirus (MCV) comprising a nucleic acid sequence having at least 60% identity with SEQ ID NO:5 (exon 2 of large T antigen, LT) and wherein said VLP has been isolated from a patient, preferably a patient suffering from Merkel Cell Carcinoma (MCC) in an episomal form or integrated in the patient's genome.

In another aspect, the invention also relates to an isolated nucleic acid selected from the group consisting of a nucleic acid having at least 99.4% identity with SEQ ID NO:1, a nucleic acid having at least 99.5% identity with SEQ ID NO:2, a nucleic acid having at least 99.2% identity with SEQ ID NO:4, a nucleic acid having at least 99.4% identity with SEQ ID NO:5, a nucleic acid having at least 99.5% identity with SEQ ID NO:7, a nucleic acid having at least 99.5% identity with SEQ ID NO:9 and a nucleic acid having at least 99.5% identity with SEQ ID NO:11.

In another aspect, the invention relates to a isolated polypeptide selected from the group consisting of an amino acid sequence having at least 99.6% identity with SEQ ID NO:3, an amino acid sequence having at least 98.6% identity with SEQ ID NO:6, an amino acid sequence having at least 99.4% identity with SEQ ID NO:8, an amino acid sequence having at least 99.3% identity with SEQ ID NO:10 and an amino acid sequence having at least 99.0% identity with SEQ ID NO:12, or a fragment of said polypeptide having at least 99.6% identity with the corresponding fragments of SEQ ID NO:3, SEQ ID NO:6, SEQ ID NO:8, SEQ ID NO:10 and SEQ ID NO:12, respectively.

In yet another aspect, the invention relates to an anti-MCV agent wherein said anti-MCV agent is a molecule which specifically interacts with a nucleic acid or a polypeptide as described above.

In particular, the invention pertains to an antibody which specifically recognizes an antigen comprised between amino acids 456 to 817 of SEQ ID NO: 6.

The invention also relates to methods and kits for detecting a VLP as defined above and to methods and kits for predicting the risk of developing an MCV-associated disease in a patient or for diagnosing an MCV-associated disease in a patient comprising the step of detecting a VLP as defined above in a tissue sample obtained from said patient.

The invention also relates to a method for identifying an agent that attenuates MCV infection comprising the step of exposing a target DNA to a polypeptide as defined above in the presence or absence of a test compound.

The invention also relates to a pharmaceutical composition comprising one or several of the elements as defined above and a pharmaceutically acceptable carrier.

### DETAILED DESCRIPTION OF THE INVENTION

The inventors have identified a novel clone of Merkel Cell Polyomavirus (MCV) associated with Merkel cell Carcinoma (MCC), which presents several differences compared to the known clones of MCV. This clone is identified as MCV-IC13.

The genome of said MCV IC-13 consists in the nucleic acid sequence as set forth in SEQ ID NO:1.

The overall identity between MCV IC-13 and the MCV clones of the prior art is 99.35% with MCV350 and 96% with MCV339. However, one principal difference between said clones resides in the fact that the clone of the MCV possesses a full length LT antigen.

The genome of MCV IC-13 contains 5 genes (see Table 1):
the nucleic acid sequence spanning from positions 196 to 756 of SEQ ID NO:1 (SEQ ID NO:2), which encodes the small T antigen (ST) having the amino acid sequence as set forth in SEQ ID NO:3;
the nucleic acid sequences spanning from positions 196 to 429 of SEQ ID NO:1 (SEQ ID NO:4) and from positions 861 to 3080 of SEQ ID NO:1 (SEQ ID NO:5), which correspond to exons 1 and 2 respectively of the large T antigen (LT).
The amino acid sequence of LT is the sequence as set forth in SEQ ID NO: 6;
the nucleic acid sequences spanning from positions 3156 to 4427 of SEQ ID NO:1 (SEQ ID NO:7), from positions 4393 to 5118 of SEQ ID NO: 1 (SEQ ID NO:9) and from positions 4393 to 4983 of SEQ ID NO:1 (SEQ ID NO:11), encoding the three structural viral proteins VP1, VP2 and VP3, respectively.
The amino acid sequences of VP1, VP2 and VP3 are respectively set forth as SEQ ID NO: 8, SEQ ID NO: 10 and SEQ ID NO: 12.

As can be seen in Table 1, the MCV clone identified by the inventors, named MCV-IC13, presents several differences with the previously identified clones, MCV 350 and MCV 339. In particular:
the LT protein as set forth in SEQ ID NO:6 is a full length protein of 817 amino acids, whereas that of MCV350 contains a stop codon at position 259 and that of MCV339 contains a deletion which also results in a truncated protein of 469 amino acids;
the sequence of the ST protein as set forth in SEQ ID NO:3 differs by one amino acid from those of MCV350 and MCV339;
the sequences of VP1, VP2 and VP3 viral proteins (SEQ ID NO:8, SEQ ID NO:10 and SEQ ID NO:12, respectively) contains several point mutations compared to their homologues in MCV350 and MCV339;

TABLE 1
Comparison of the MCV-IC13 clone with clones MCV350 and MCV339

| Sequence of | Nucleic acid | | | | Protein | | |
|---|---|---|---|---|---|---|---|
| MCV-IC13 | SEQ ID | | % identity with MCV | % identity with | SEQ ID | % identity with MCV | % identity with MCV |
| clone | NO: | positions | 350 | MCV 339 | NO: | 350 | 339 |
| Complete | NO: 1 | 1-5387 | 99.35% | 96% | | N/A | N/A |
| genome | | | | | | | |
| ST | NO: 2 | 196-756 | nd | nd | NO: 3 | 99.5% | 99.5% |
| LT | | | | | NO: 6 | 98.5% | N/A |
| exon 1 | NO: 4 | 196-429 | 98.7% | 99.10% | | | |
| exon 2 | NO: 5 | 861-3080 | 99.3% | 91.10% | | | |
| VP1 | NO: 7 | 3156-4427 | nd | nd | NO: 8 | 98.6% | 99.3% |
| VP2 | NO: 9 | 4393-5118 | nd | nd | NO: 10 | 98.8% | 99.2% |
| VP3 | NO: 11 | 4393-4983 | nd | nd | NO: 12 | 98.9% | 98.9% |

nd: not determined
N/A: not applicable (the percentage could not be calculated, due to the presence of several proteins encoded by SEQ ID NO: 1 or due to a large deletion, which would bias the calculation in the case of LT of MCV 339)

To determine the percent identity of two nucleic acid sequences, the sequences are aligned for optimal comparison. For example, gaps can be introduced in the sequence of a first nucleic acid sequence for optimal alignment with the second nucleic acid sequence. The nucleotides at corresponding nucleotide positions are then compared. When a position in the first sequence is occupied by the same nucleotide as at the corresponding position in the second sequence, the nucleic acids are identical at that position. The percent identity between the two sequences is a function of the number of identical nucleotides shared by the sequences.

Hence % identity=[number of identical nucleotides/total number of overlapping positions]×100. The percentage of sequence identity is thus calculated according to this formula, by comparing two optimally aligned sequences over the window of comparison, determining the number of positions at which the identical nucleic acid base (e.g., A, T, C, G) occurs in both sequences to yield the number of matched positions (the “number of identical positions” in the formula above), dividing the number of matched positions by the total number of positions in the window of comparison (e.g. the window size) (the “total number of overlapping positions” in the formula above), and multiplying the result by 100 to yield the percentage of sequence identity.

In this comparison, the sequences can be the same length or may be different in length. Optimal alignment of sequences for determining a comparison window may be conducted by the local identity algorithm of Smith and Waterman (1981), by the identity alignment algorithm of Needleman and Wunsh (1972), by the search for similarity via the method of Pearson and Lipman (1988), by computerized implementations of these algorithms (GAP, BESTFIT, FASTA and TFASTA in the Wisconsin Genetics Software Package Release 7.0, Genetic Computer Group, 575, Science Drive, Madison, Wis.), or by inspection.

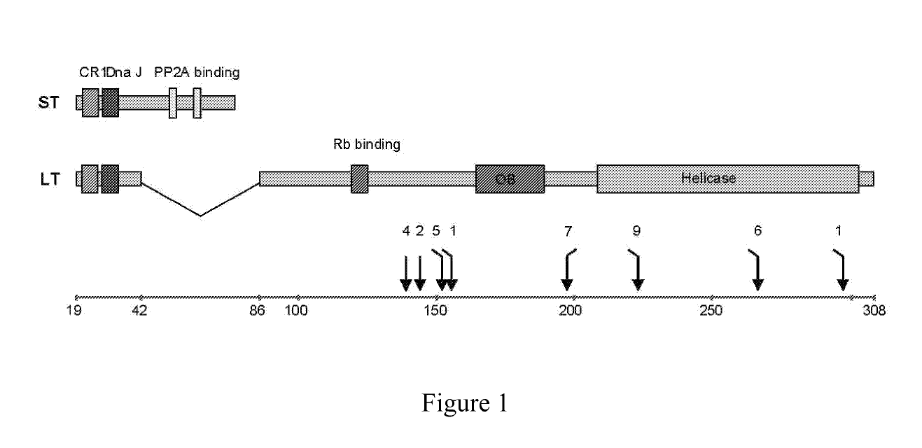

Without wishing to be bound by theory, it is believed that the differences in the above sequences result in the physiopathology of clone MCV IC-13 being different from that of any known MCV clone. In particular it is believed that, in MCV IC-13, Small T (ST) and Large T antigens have different biological properties from those of the prior art. Of particular interest, the LT antigen of MCV IC-13 is unique in that it possesses a conserved helicase domain (see FIG. 1).

### Viral-Like Particles (VLP) of the Invention

In one embodiment, the invention relates to a virus-like particle (VLP) wherein said VLP is derived from a polyomavirus, preferably a Merkel cell Polyomavirus (MCV), comprising a nucleic acid sequence having at least 60% identity with SEQ ID NO:5 (exon 2 of large T antigen, LT) and wherein said VLP has been isolated from a patient, preferably a patient suffering from Merkel Cell Carcinoma (MCC) in an episomal form or integrated in the patient's genome.

In a preferred embodiment, said VLP comprises a nucleic acid sequence having at least 65% identity with SEQ ID NO:5, preferably at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identity with SEQ ID NO:5, even more preferably at least 99.5%, at least 99.6%, at least 99.7%, at least 99.8%, at least 99.9%, at least 99.95% identity with SEQ ID NO:5.

As used herein, the expression “viral-like particle” or “VLP” encompasses both viral particles and particles that resemble the virus from which they were derived but lack viral nucleic acid. VLP according to the invention therefore comprise at least a viral envelope. VLP can therefore be infectious (when they comprise viral nucleic acid) or non-infectious particles (when they are devoid of nucleic acid).

As used herein, the term “polyomavirus” has its general meaning in the art. Polyomaviruses are DNA-based (double-stranded DNA, ˜5000 base pairs, circular genome), small (40-50 nanometers in diameter), and icosahedral in shape, and do not have a lipoprotein envelope. They are potentially oncogenic (tumor-causing); they often persist as latent infections in a host without causing disease, but may produce tumors in a host of a different species, or a host with an ineffective immune system. The name polyoma refers to the viruses' ability to produce multiple (poly-) tumors (-oma).

As used herein, the expression “Merkel Cell Polyomavirus” or “MCV” has its general meaning in the art. MCV is a human polyomavirus, first discovered in 2008, which is highly divergent from the other human polyomaviruses and is most closely related to murine polyomavirus.

As used herein, the term “patient” can include human patients as well as animals. In this respect, the diagnostic and therapeutic methods can be performed in the veterinary context, i.e., on domestic animals, particularly mammals (e.g., dogs, cats, etc.) or agriculturally-important animals (e.g., horses, cows, sheep, goats, etc.) or animals of zoological importance (apes, such as gorillas, chimpanzees, and orangutans, large cats, such as lions, tigers, panthers, etc., antelopes, gazelles, and others). In a preferred embodiment, said patient is a mammalian, preferably a primate, even more preferably a human patient.

The VLP of the invention can be isolated from a patient. In other terms, the VLP according to the invention can be isolated from a tissue sample of a patient. Within said tissue sample, the VLP can be present either in an episomal form or integrated in the patient's genome.

The skilled person in the art knows how to isolate a VLP from a patient and can readily determine whether said VLP is present as an episome, i.e. as a separate nucleic acid molecule, or whether it is integrated into one of the patient's chromosomes, using standard techniques in the art.

In a preferred embodiment, said VLP is isolated from a patient suffering from Merkel Cell Carcinoma.

In one embodiment, the VLP of the invention further comprises a polypeptide of at least 470 amino acids having at least 60% identity with SEQ ID NO:6 (LT polypeptide) over said at least 470 amino acids.

As used herein, the terms “polypeptide” or “protein” can be used interchangeably and have their general meaning in the art. Polypeptides or proteins comprise two or more amino acid residues linked together by a peptide bond.

In a preferred embodiment, said polypeptide has at least 70%, preferably at least 80%, 85%, 90%, 91%, 92%, 93%, 94%, 95%, 96%, 97%, 98%, 99% identity with SEQ ID NO: 6 over the length of said polypeptide, even more preferably at least 99.5%, at least 99.6%, at least 99.7%, at least 99.8%, at least 99.9%, at least 99.95% identity with SEQ ID NO:6 over the length of said polypeptide.

In a preferred embodiment, said polypeptide comprises at least 500 amino acids, even more preferably at least 600, 700, 750, 760, 770, 780, 790, 800, 810, 811, 812, 813, 814, 815, 816 or 817 amino acids.

Thus, in one embodiment, the VLP comprises a polypeptide of about 817 amino acids having at least 60% identity with SEQ ID NO:6, preferably at least 70%, preferably at least 80%, 85%, 90%, 91%, 92%, 93%, 94%, 95%, 96%, 97%, 98%, 99% identity with SEQ ID NO: 6 over the length of said polypeptide, even more preferably at least 99.5%, at least 99.6%, at least 99.7%, at least 99.8%, at least 99.9%, at least 99.95% identity with SEQ ID NO:6.

In one embodiment, the VLP according to the invention further comprises at least one nucleic acid selected from the group consisting of the nucleic acids having:
at least 99.4% identity with SEQ ID NO: 5 (exon 2 of LT);
at least 99.2% identity with SEQ ID NO:4 (exon 1 of LT);
at least 99.5% identity with SEQ ID NO:2 (ST);
at least 99.5% identity with SEQ ID NO:7 (VP1);
at least 99.5% identity with SEQ ID NO:9 (VP2);
at least 99.5% identity with SEQ ID NO:11 (VP3) and
at least 99.4% identity with SEQ ID NO:1 (complete genome of MCV-IC13).

In one embodiment, the VLP according to the invention comprises a nucleic acid having at least 99.4% identity with the sequence as set forth in SEQ ID NO:5 (exon 2 of LT).

In a preferred embodiment, said sequence has at least 99.5% identity with the sequence as set forth in SEQ ID NO:5, even more preferably at least 99.6%, 99.7%, 99.8%, 99.9% or 99.95% identity with the sequence as set forth in SEQ ID NO: 5.

In one embodiment, the VLP according to the invention comprises a nucleic acid having at least 99.2% identity with the sequence as set forth in SEQ ID NO: 4 (exon 1 of LT).

In a preferred embodiment, said sequence has at least 99.3% identity with the sequence as set forth in SEQ ID NO:4, even more preferably at least 99.4%, 99.5%, 99.6%, 99.7%, 99.8%, 99.9% or 99.95% identity with the sequence as set forth in SEQ ID NO: 4.

In one embodiment, the VLP according to the invention comprises a nucleic acid having at least 99.4% identity with the sequence as set forth in SEQ ID NO: 1 (complete genome of MCV-IC13).

In a preferred embodiment, said nucleic acid has at least 99.5% identity with the sequence as set forth in SEQ ID NO: 1, even more preferably 99.6%; 99.7%; 99.8%; 99.9% or 99.95% identity with the sequence as set forth in SEQ ID NO: 1.

Typically said isolated virus is not selected from the group consisting of MCV350, MCV339, MCV352 and MCV MKL1.

In one embodiment, the genome of said isolated virus consists essentially in SEQ ID NO:1.

As used herein, the expression “consists essentially in” means that, out of a nucleic acid sequence of several thousands of base pairs, minor differences in sequence are tolerated, so long as they do not impede in the function of the nucleic acid.

### Nucleic Acids and Polypeptides of the Invention

The invention also relates to an isolated nucleic acid selected from the group consisting of a nucleic acid having at least 99.4% identity with SEQ ID NO:1, a nucleic acid having at least 99.5% identity with SEQ ID NO:2, a nucleic acid having at least 99.2% identity with SEQ ID NO:4, a nucleic acid having at least 99.4% identity with SEQ ID NO:5, a nucleic acid having at least 99.5% identity with SEQ ID NO:7, a nucleic acid having at least 99.5% identity with SEQ ID NO:9 and a nucleic acid having at least 99.5% identity with SEQ ID NO:11.

In one embodiment, the invention relates to a nucleic acid having at least 99.4% identity with SEQ ID NO: 1.

In a preferred embodiment, said sequence has at least 99.5% identity with the sequence as set forth in SEQ ID NO: 1, even more preferably 99.6%; 99.7%; 99.8%; 99.9% or 99.95% identity with the sequence as set forth in SEQ ID NO: 1.

In one embodiment, the invention relates to a nucleic acid having at least 99.2% identity with the sequence as set forth in SEQ ID NO: 4 (exon 1 of LT).

In a preferred embodiment, said sequence has at least 99.3% identity with the sequence as set forth in SEQ ID NO:4, even more preferably at least 99.4%, 99.5%, 99.6%, 99.7%, 99.8%, 99.9% or 99.95% identity with the sequence as set forth in SEQ ID NO: 4.

In one embodiment, the invention relates to a nucleic acid having at least 99.4% identity with the sequence as set forth in SEQ ID NO:5 (exon 2 of LT).

In a preferred embodiment, said sequence has at least 99.5% identity with the sequence as set forth in SEQ ID NO:5, even more preferably at least 99.6%, 99.7%, 99.8%, 99.9% or 99.95% identity with the sequence as set forth in SEQ ID NO: 5.

The invention also relates to a isolated polypeptide selected from the group consisting of an amino acid sequence having at least 99.6% identity with SEQ ID NO:3, an amino acid sequence having at least 98.6% identity with SEQ ID NO:6, an amino acid sequence having at least 99.4% identity with SEQ ID NO:8, an amino acid sequence having at least 99.3% identity with SEQ ID NO:10 and an amino acid sequence having at least 99.0% identity with SEQ ID NO:12, or a fragment of said polypeptide having at least 99.6% identity with the corresponding fragments of SEQ ID NO:3, SEQ ID NO:6, SEQ ID NO:8, SEQ ID NO:10 and SEQ ID NO:12, respectively.

Typically, said fragments are significantly different from the homologous fragments in MCV 350 and MCV 339.

In a preferred embodiment, the invention relates to a fragment of polypeptide having at least 98.6% identity with SEQ ID NO:6, wherein said fragment encompasses the amino acids 456 to 817 of SEQ ID NO:6. Amino acids 456 to 817 of SEQ ID NO:6 are absent from both MCV350 and MCV339 due to truncation of the LT protein. This fragment comprises the helicase domain of the LT antigen.

### Anti-MCV Agents of the Invention

Also provided herein are anti-MCV agents. As used herein, the expression “anti-MCV agent” refers to a molecule that can be used to recognize a VLP according to the invention. In particular, said anti-MCV agents are useful for discriminating between the MCV-IC13 clone of the invention (and VLP according to the invention derived therefrom) and the MCV clones of the prior art.

In a preferred embodiment, said anti-MCV agent is an agent which can further be used to attenuate an MCV infection.

The invention therefore relates to an anti-MCV agent wherein said anti-MCV agent is a molecule which specifically interacts with a nucleic acid or a polypeptide as described above.

In one embodiment, detection of a nucleic acid is carried out by hybridization under stringent conditions with a probe that is selectively hybridizable with of SEQ ID NO:1.

Within the context of the present invention, a nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm−5° C. (5° C. below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while high stringency conditions are used to identify sequences having about 80% or more sequence identity with the probe. This is especially true for polynucleotides having a minimum of from about 18-22 nucleic acids, but those of ordinary skill in the art are also able to apply these principals to larger or smaller polynucleotides.

Moderate and high stringency hybridization conditions are well known in the art (see, for example, Sambrook et al. Molecular Cloning: A Laboratory Manual (Second Edition), Cold Spring Harbor Press, Plainview, N.Y., 1989, especially chapters 9 and 11; and Ausubel F M et al. Current Protocols in Molecular Biology, John Wiley & Sons, New York, N.Y., 1993). An example of high stringency conditions includes hybridization at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C.

In a preferred embodiment, said probe has at least 99.4% identity with SEQ ID NO:5 (exon 5 of LT).

The invention also relates to an anti-agent which specifically recognizes a polypeptide as defined above.

The molecules which specifically interact with a polypeptide of the invention may be an antibody that may be polyclonal or monoclonal, preferably monoclonal. In another embodiment, the molecule which specifically interacts with a polypeptide of the invention may be an aptamer.

Polyclonal antibodies of the invention can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred.

Monoclonal antibodies of the invention can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see e.g. U.S. Pat. No. 4,946,778) can be adapted to produce single chain antibodies. Antibodies useful in practicing the present invention also include fragments including but not limited to F(ab′)2 fragments, which can be generated by pepsin digestion of an intact antibody molecule, and Fab fragments, which can be generated by reducing the disulfide bridges of the F(ab′)2 fragments. Alternatively, Fab and/or scFv expression libraries can be constructed to allow rapid identification of fragments having the desired specificity. For example, phage display of antibodies may be used. In such a method, single-chain Fv (scFv) or Fab fragments are expressed on the surface of a suitable bacteriophage, e.g., M13. Briefly, spleen cells of a suitable host, e.g., mouse, that has been immunized with a protein are removed. The coding regions of the VL and VH chains are obtained from those cells that are producing the desired antibody against the protein. These coding regions are then fused to a terminus of a phage sequence. Once the phage is inserted into a suitable carrier, e.g., bacteria, the phage displays the antibody fragment. Phage display of antibodies may also be provided by combinatorial methods known to those skilled in the art. Antibody fragments displayed by a phage may then be used as part of an immunoassay.

Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consist of conformationally constrained antibody variable regions displayed by a platform protein, such as E. coli Thioredoxin A, that are selected from combinatorial libraries by two hybrid methods (Colas et al., 1996).

In one aspect, the invention relates to an antibody which specifically recognizes the non-truncated LT protein. As used herein, the expression “specifically recognizes the non-truncated LT protein” means that said antibody binds to a non-truncated LT protein but does not bind to (or binds with a significantly lower affinity, i.e. an affinity lower by a factor of at least 5, preferably by a factor of at least 10, even more preferably by a factor of at least 100) to the truncated LT proteins of MCV clones of the prior art. Typically, the antibody of the invention binds to the LT protein of clone MCV-IC13, but not to the LT protein of clones MCV350 or MCV339.

In one aspect, the invention also relates to an antibody which specifically recognizes the LT protein as set forth in SEQ ID NO:6 and fragments thereof, wherein said fragments comprises at least 8 successive amino acids among amino acids 456 to 817 of SEQ ID NO: 6.

In a particular embodiment, the invention relates to an antibody which specifically recognizes an antigen comprised between amino acids 456 to 817 of SEQ ID NO: 6.

In another embodiment, the invention relates to an antibody which specifically recognizes a conformational epitope wherein said conformational epitope is partly comprised of residues located between amino acids 456 to 817 of SEQ ID NO: 6. In this embodiment, the conformational epitope may comprise other residues located elsewhere in the LT protein, but which are in proximity with said residues located between amino acids 456 to 817 of SEQ ID NO: 6 in the 3D structure of the LT protein.

In one embodiment, said anti-MCV agent wherein said anti-MCV agent inhibits the expression and/or the activity of at least one nucleic acid of the invention or at least one polypeptide of the invention.

### Detection Methods and Kits According to the Invention

The invention also relates to methods and kits for detecting a VLP as defined above and to methods and kits for predicting the risk of developing an MCV-associated disease in a patient or for diagnosing an MCV-associated disease in a patient comprising the step of detecting a VLP as defined above in a tissue sample obtained from said patient.

The anti-MCV agents described above are useful for the following detection methods.

In one aspect, the invention relates to a method for detecting a VLP according to the invention, comprising the step of detecting a nucleic acid or a polypeptide as defined above.

In a preferred embodiment, the invention relates to a method for detecting a VLP according to the invention, comprising the step of detecting an LT protein with an antibody that specifically recognizes full length LT. Said antibody can be for instance and antibody directed against an epitope comprised between amino acids 456 to 817 of SEQ ID NO: 6. This sequence is absent from clones MCV350 and MCV339.

In one aspect, the invention provides a method for predicting the risk of developing an MCV-associated disease in a patient comprising the step of detecting a VLP as defined above in a tissue sample obtained from said patient.

In one aspect, the invention provides a method for diagnosing an MCV-associated disease in a patient comprising the step of detecting a VLP as defined above in a tissue sample obtained from said patient.

Typically said tissue sample may be a blood sample, a urine sample or a biopsy. In a preferred embodiment said tissue sample is a biopsy, preferably a skin biopsy.

As used herein, the expression “MCV-associated disease” refers to a disease in which MCV is a causative agent. An MCV-associated disease can be primary MCV infection or a cancer (such as Merkel cell carcinoma, small cell lung carcinoma, or other carcinoma associated with MCV infection)

In a preferred embodiment said cancer is Merkel Cell Carcinoma (MCC).

In another embodiment, said cancer is not Merkel Cell Carcinoma (MCC).

The invention also relates to a kit for diagnosis an MCV-associated disease in a patient comprising an anti-MCV agent as defined above.

Typically, said kit can comprise an antibody which specifically recognizes an antigen comprised between amino acids 456 to 817 of SEQ ID NO: 6 and means for revealing said antibody.

### Screening Method of the Invention

The invention also relates to a method for identifying an agent that attenuates MCV infection.

In another embodiment, the invention provides a method of identifying an agent that attenuates MCV infection, comprising the step of exposing a target DNA to a polypeptide as defined above in the presence or absence of a test compound.

In this context, attenuation can involve the reduction of likelihood of infection, or reduction in magnitude. In some applications, the reduction can amount to complete prophylaxis. In accordance with this method, target DNA is exposed to a MCV polypeptide (e.g., VP1, VP2, VP3, LT or ST). The target DNA should include a sequence to which the MCV polypeptide can specifically bind relative a negative control DNA. The assay is conducted in the presence of a test agent, which is a putative agent under investigation to assess whether it can attenuate the MCV infection. Thus, the MCV polypeptide and the target DNA are exposed to each other under conditions which, except for the test substance, are suitable for the MCV polypeptide and target DNA to bind. It will be understood that, as a result of this assay, the ability of the test substance to attenuate binding of the MCV protein to the target DNA identifies the test substance as a candidate agent for use as an anti-MCV therapeutic agent. An example of this type of assay is a gel-shift assay, which is known to those of ordinary skill in the art. Also, while the test agent can be identified as a candidate MCV therapeutic agent by this method, other tests likely will be needed to assess whether the agent is safe and effective for clinical use.

### Methods of Treatment and Pharmaceutical Compositions of the Invention

In some aspects the invention involves prophylactic and therapeutic methods against an MCV-associated disease.

The invention also relates to a pharmaceutical composition comprising a virus, a nucleic acid and/or a protein of the invention.

The invention therefore relates to a pharmaceutical composition comprising one or several of the elements as defined above and a pharmaceutically acceptable carrier.

In particular, the invention relates to a pharmaceutical composition comprising:
a VLP according to the invention and/or
a nucleic acid according to the invention and/or
a polypeptide according to the invention and/or
an anti-MCV agent according to the invention and/or
an agent that attenuates MCV infection obtainable by the method as defined above
   and a pharmaceutically acceptable carrier.

Suitable pharmaceutical compositions can be formulated for delivery by oral, nasal, transdermal, parenteral, or other routes by standard methodology. In this respect, the excipient can include any suitable excipient (e.g., lubricant, diluent, buffer, surfactant, co-solvent, glidant, etc.) known to those of ordinary skill in the art of pharmaceutical compounding (see, e.g., “Handbook of Pharmaceutical Excipients” (Pharmaceutical Press), Rowe et al, 5th Ed. (2006)).

In another aspect, the invention relates to prophylactic and therapeutic methods against MCV-associated diseases. In this context, the MCV-associated disease can be primary MCV infection or a carcinoma (such as Merkel cell carcinoma, small cell lung carcinoma, or other carcinoma associated with MCV infection). For example, the invention provides a method of vaccinating a patient against an MCV-associated disease. In accordance with this method, a patient is vaccinated with MCC DNA and/or a MCV polypeptide under conditions suitable for the patient to generate an immune response to the MCV DNA and/or MCC polypeptide. A preferred agent for serving as the vaccine is a polypeptide comprising at least 10, and preferably at least the majority of, contiguous amino acids from amino acids 456 to 817 of SEQ ID NO: 6. Another preferred agent is a VLP as herein described. Indeed, rabbits and mice immunized with an MCV VLP can exhibit very high anti-MCV antibody responses, with 50% neutralizing titers in the million-fold dilution range. It will be understood that VLP could be combined with other viral subunit vaccines such as the current vaccines against hepatitis B virus and human papillomavirus, for combined vaccination protocols.

In another aspect, the invention provides a method for treating a patient suffering from an MCV-associated disease involving adoptive immunotherapy. In accordance with this method, a population of T lymphocytes is first obtained from the patient. Thereafter, the population of T lymphocytes is exposed ex vivo to an MCV polypeptide, including a virus (such as described herein) under conditions suitable to activate and expand the population of T lymphocytes. For example, the T lymphocytes can be exposed to cells in vitro, which express an MCV protein (e.g., having been transfected with an expression cassette encoding the MCV polypeptide). A preferred MCV protein includes at least 10, and preferably at least the majority of, contiguous amino acids from amino acids 456 to 817 of SEQ ID NO: 6. In other aspects, the method can be practices using standard techniques (see, e.g., June, J. Clin. Invest., 117(6) 1466-76 (2007)). After they have been activated, at least some of the T lymphocytes are re-introduced into the patient. Such a method can attenuate the severity of the MCV-associated disease within the patient. It should be understood that the method need not eradicate the MCV-associated disease within the patient to be effective as a therapy. The method can be deemed effective if it lessens symptoms, improves prognosis, or augments other modes of therapy if used adjunctively.

It is believed that the newly-discovered MCV should respond to agents that interfere with the replication of other polyomaviruses. Thus, the invention provides a method of treating an MCV-associated disease by administering such an agent to a patient suffering from an MCV-associated disease. As noted, the MCV-associated disease can be primary MCV infection, Merkel cell carcinoma, small cell lung carcinoma, or another carcinoma that is caused by MCV. It is believed that the administration of some such agents can attenuate the severity of the MCV-associated disease within the patient. Examples of such agents are cidofovir and vidarabine, and other agents that interfere with polyomavirus replication known to those of ordinary skill may be useful in treating such conditions as well. Additional agents include interferons and mTOR inhibitors (e.g., sirolimus and tacrolimus).

The invention will be further described by the following examples, which are not intended to limit the scope of the protection defined by the claims.

FIG. 1: Schema of the main functional domains of ST and LT proteins.

The bottom line shows the corresponding nucleotide positions in the MCV genome. Arrows indicate the position of the interruption of integrated viral DNA sequences determined by DIPS-PCR at the 3′ end ( [symbol] ) or the 5′ end ( [symbol] ) of the viral genome. ↓ refers to mutation leading to the stop codon identified by sequencing. The numbers above the arrows correspond to case number.

CR1: Conserved Region 1; OBD: Origin Binding Domain

### EXAMPLES

### Methods

### Cases and Tumour Specimens

Ten cases of MCC were accumulated from 1996 to 2007. For 9 cases, a tumour specimen was fixed in formalin for histological analysis and another specimen frozen in liquid nitrogen then kept at −70° C. for molecular studies. In one case, a patient with MCC from the nasal septum (N^{o} 4), only a fine needle aspiration product of a supra-clavicular lymph node was available for cytological analysis and DNA extraction. Twelve tumour specimens were analysed from these 10 patients, corresponding to primary tumours in 6 cases, 3 skin metastases and 3 lymph node metastases.

According to the French regulation, patients were informed of researches performed using the biological specimens obtained during their treatment and did not express opposition.

### Histological Analysis

Tumours were analysed according to standard histological procedure. Histological reports specified the architectural pattern as solid/cohesive (massive or trabecular) or diffuse/discohesive [3] Immunohistochemistry was performed to confirm the diagnosis using antibodies directed against chromogranin A (clone DAK-A3, dilution: 1/200; Dako, Glostrup, Denmark) and synaptophysin (clone SY38, dilution 1/40; Dako), markers expressed by virtually all MCCs [4, 17]. Reactivity was scored as follows: 1: <10% of reactive cells; 2: 10-50%; 3: >50%. Detection of cytokeratin intermediate filaments was performed using pan anti-cytokeratins (clone KL1, dilution 1/200; Beckman Coulter, Villepinte, France). Staining was revealed by the Avidin Biotin technique, using DAB as a chromogen (Dako).

### MCV350 DNA Sequences Screening and Viral Load

MCV350 sequences were detected by PCR amplification with primers MCV_ST_A and MCV_ST_B specific for the ST sequences (size product 165 bp), MCV_LT_C and MCV_LT_D for the LT sequences (162 bp), and MCV_VP1_A and MCV_VP1_B for VP1 gene (204 bp). The viral load was obtained by amplification of DNA (10 ng) with 600 nM of each MCV_LT_C and MCV_LT_D primers in the SYBR Green PCR master mix (Applied Biosystems, Courtaboeuf, France), using the standard cycling conditions of 10 min at 95° C. and 40 cycles (15 s at 95° C., 1 min at 60° C.). Amplification of a genomic DNA sequence ZNF277 (7q31.1) with primers IC5A and IC5B was used as DNA quality control and reference for two copies of DNA sequences per cell. Viral copy number was estimated by quantitative PCR using a delta-delta Ct method [18].

In non MCC tumours, a total of 1277 DNA specimens from tumours of various histological types and organs were obtained from the DNA bank of the Institut Curie. DNA quality control assessed by amplification of the ZNF277 DNA sequences showed that DNA quality was insufficient in 36 cases which were discarded from the study. The 1241 remaining specimens were analysed for MCV sequences using MCV_LT_C and MCV_LT_D primers designed in the 5′ part of the LT sequences. See supplementary file for primer sequences.

### MCV Cloning and FISH Experiments

The whole viral genome could be amplified in one case (n° 5) using primers MCV-U2 and MCV-L2 located at bases 5283 and 5282 of the MCV genome, respectively. DNA (250 ng) was amplified by PCR (final volume 25 μl) using the Expand 20 kb^{plus} PCR System (Roche Applied Science, Meylan, France). The viral genome (5387 bp) was then cloned in the pCR®-XL-TOPO® vector (Invitrogen, Carlsbad, Calif. 92008) and sequenced. One of the clones isolated (MCV-IC13) proved to encompass the whole viral genome without any mutation likely to interrupt the coding sequences. This genome was used for fluorescent in situ hybridization (FISH) experiments. DNA was labelled by Nick translation using the BioNick™ Labelling System (Invitrogen) with biotinylated dATP. Hybridization was performed on frozen histological sections. The slides were analysed using a Leica DMRB microscope fitted with Quips (Visys, Downers Grove, Ill. 60515) Image Capture System.

### Viral Integration Sites

The DIPS-PCR technique, which allows the amplification of genomic viral-cellular junctions [19], was used to investigate the integration sites of MCV in MCC. After tumour DNA digestion with restriction enzyme Taq1, enzyme-specific adapters were ligated to the restriction fragments. The ligation products obtained were subjected to PCR amplification which consisted of a first round of linear PCR with a viral specific primer a, followed by a second round of exponential PCR with a viral specific primer b, internal to the previous one, and a second primer AP1 specific for the adapter. The 3′ viral-cellular DNA junctions were detected with primers f_a and f_b and the cellular-5′ viral DNA junctions with primers r_a and r_b. PCR products were excised from an agarose gel, purified and sequenced (see supplementary file a for primer sequences). Sequences were submitted to database (UCSC Genome Browser website; Working Draft March 2006) for genomic localisation.

### Viral Gene Expression

Total RNA was isolated using Trizol reagent (Invitrogen). DNAse digestion using the Nucleospin RNA/Protein kit (Macherey-Nagel, Hoerdt, France) was performed. Total RNA (1 μg) was reverse transcribed (RT+) using the GeneAmp RNA PCR Core Kit (Applied Biosystems). For each sample, a negative control without reverse transcriptase (RT−) was performed to verify the absence of contaminating DNA. PCRs were performed in parallel on the RT+ and RT− products. One hundredth of the RT+ or RT− product was used for each PCR reaction (final volume of 25 μl), in the presence of 600 nM of each specific primer and in the SYBR Green PCR master mix (Applied Biosystems). Primers MCV_ST_A and MCV_ST_B were used for MCV ST expression, MCV_LT_C and MCV_LT_D for LT expression. MCV_ST_B primer was designed in the spliced LT sequences and thus allows the amplification of ST sequences only. A12 and A13 primers were used for the TATA Box Binding Protein (TBP) gene as a reference for gene expression level. PCR amplifications were performed in an ABI PRISM 7500 (Applied Biosystem). MCV350 mRNA expression levels were directly compared to TBP expression using the delta-delta Ct method [18].

### MYC and IL20RA Gene Expression.

PCR with primers A227 and A228 for MYC expression and IL20RA_B and IL20RA_C for IL20RA were performed in the conditions previously described for the MCV350 genes.

### Array-CGH.

Tumour cellularity of the samples was verified to be >60%. Tumour DNA was prepared using DPNII digestion (Ozyme, St Quentin-en-Yvelines, France), and purification on QIAquick column (Qiagen, Courtaboeuf, France). Reference and test DNAs were labelled with Cy3 and Cy5 cyanine dyes respectively (PerkinElmer, Courtaboeuf, France) using the BioPrime random priming labelling kit (Invitrogen). Reference and test DNA were precipitated together with human Cot-1 DNA (Invitrogen), resuspended in hybridization buffer, and denatured. The DNA was hybridised onto a genome-wide DNA microarray consisting of 5K BAC clones spotted in triplicate, with a 1 Mbase resolution (CIT/INSERM U830/IntegraGen). Slides were scanned using an Axon GenePix 4000B scanner (Molecular Devices, Sunnyvale, Calif.). Image analysis was performed with the Axon GenePix 5•1 software (Molecular Devices). The data was visualized using the VAMP software [20].

### Results

### Patients and Tumours

Ten cases of Merkel cell carcinoma (MCC) in 8 male and 2 female patients with a mean age of 79.1 (63-85) were studied (table 2). All primary tumours were dermal, localised on the head and neck in 4 cases, the limbs in 4 cases, and the trunk in 2 cases (table 2). In one case (N^{o} 6), two cutaneous metastases were analysed in addition to primary tumour. The mean primary tumour size was 23.9 mm (10 to 45 mm) Histological analysis showed the architectural pattern to be solid in 5 cases, either trabecular (3 cases) or massive (2 cases) and discohesive/diffuse in 4 cases. This latter pattern was characterised by a proliferation of tumour cells that lack cohesion and appear individually dispersed throughout the connective tissue (data not shown). All 9 cases exhibited the co-expression of chromogranin A and synaptophysin neuro-endocrine markers, a characteristic immunophenotype of MCC (data not shown and table 2). Cytokeratins staining disclosed a dot-like immunolabelling close to the nucleus of tumour cells, corresponding to a localised aggregate of these intermediate filaments (data not shown).

TABLE 2
Clinical and histological data in Merkel cell carcinoma

| | | | | | | histology | | |
|---|---|---|---|---|---|---|---|---|
| | | | localisation | | size | | immunophenotype | |
| Case | age | Sex | primary | metastasis | (mm) | pattern | chromogranin | synaptophysin |
| 1 | 82 | M | eyelid | cervical LN* | 30 | discohesive/diffuse | 3 | 1 |
| 2 | 72 | M | forearm* | | 10 | discohesive/diffuse | 2 | 2 |
| 3 | 84 | M | thigh | inguinal LN* | 27 | discohesive/diffuse | 3 | 2 |
| 4 | 79 | F | nose | cervical LN* | 30 | solid/trabecular | ND | ND |
| 5 | 81 | M | ankle | leg* | 11 | solid/trabecular | 2 | 2 |
| 6a | 80 | M | wrist* | | 21 | solid/massive | 3 | 3 |
| 6b | | | | trunk* | — | ND | ND | ND |
| 6c | | | | trunk* | — | ND | ND | ND |
| 7 | 82 | F | cheek* | | 15 | solid/massive | 1 | 1 |
| 8 | 85 | M | buttock* | | 30 | discohesive/diffuse | 3 | 2 |
| 9 | 63 | M | breast* | | 20 | solid/trabecular | 2 | 3 |
| 10 | 83 | M | ear* | | 45 | solid/massive | 3 | 3 |

LN: lymph node
*specimens analysed for MCV characterisation

TABLE 3
Viral and genetic data in Merkel cell carcinoma

| | | | | | | | | | | Putative | |
|---|---|---|---|---|---|---|---|---|---|---|---|
| | | | | | | | | | | target genes | |
| | | | MCV | RNA | Array-CGH | | | | | expression | |
| | MCV | Viral | expression level | | chromosome | Chromosome | | MCV | Putative | level | |
| Case | DNA | load* | small T | large T | imbalances** | insertion sites | Locus*** | breakpoints | target genes | MYC | IL20RA |
| 1 | + | 3 | 0.23 | 3.58 | +5p, −5q, −8p, | 8q24.21 | 130177462 | 1532 (3′) | MYC | 0.008 | 0.000 |
| 2 | + | 1.2 | 0.42 | 3.25 | no imbalance | 12q23.1 | 97372542 | 5202 (3′) | AX747640 | 0.007 | 0.000 |
| 3 | + | 0.6 | ND | ND | +6p, +11, +17p | 2q32.3 | 196674834 | 3925 (5′) | | — | — |
| 4 | + | 3.3 | ND | ND | −2, +6, −7, | 20q11.21 | 31474040 | 3712 (3′) | SNTA1 | — | — |
| | | | | | −10, −17 | | | | | | |
| 5 | + | 62.2 | 0.24 | 2.62 | +1p, +1q | 4q13.1 | 64683073 | 1515 (3′) | SRD5A2L2 | 0.043 | 0.073 |
| 6a | + | 3.8 | 3.16 | 21.56 | no imbalance | 3q26.33 | 183625703 | 2663 (3′) | ATP11B | 0.025 | 0.000 |
| 6b | + | ND | ND | ND | ND | 3q26.33 | 183625703 | 2663 (3′) | — | — | — |
| 6c | + | ND | ND | ND | ND | 3q26.33 | 183625703 | 2663 (3′) | — | — | — |
| 7 | + | 1.7 | 0.21 | 1.46 | +1q | 5q35.1 | 170684993 | 1978 (5′) | TLX3 | 0.034 | 0.000 |
| 8 | + | 6.3 | 0.28 | 3.86 | +11 | ? | | 3119 (5′) | | 0.012 | 0.000 |
| 9 | + | 1 | 0.33 | 3.36 | no imbalance | 6q23.3 | 137409329 | 2240 (3′) | IL20RA | 0.036 | 0.029 |
| | | | | | | | 137408299 | 3305 (5′) | | | |
| 10 | + | 10.3 | 0.39 | 5.50 | +1q, +6p, | Yq12 | 57288464 | 2980 (3′) | | 0.068 | 0.000 |
| | | | | | −6qter, +7pter | | | | | | |

*number of equivalent viral genome per cell;
**recurrent imbalances in bold;
***Working Draft march 2006

TABLE 4
MCV analysis in non MCC human tumours

| | | Nb of | MCV |
|---|---|---|---|
| Organs | Histological tumour type | cases | DNA |
| Skin | Basal cell carcinoma | 13 | — |
| | Melanoma | 13 | — |
| | Others | 2 | — |
| | Normal skin | 4 | — |
| Uterine cervix | Invasive carcinoma | | |
| | HPV positive | 26 | — |
| | HPV negative | 18 | — |
| Large bowel | Adenocarcinoma | 38 | — |
| | Others | 1 | — |
| Liver | Metastatic melanoma | 94 | — |
| | Metastatic breast carcinoma | 16 | — |
| | Others | 4 | — |
| Uveal tract | Melanoma | 45 | — |
| Ovary | Serous adenocarcinoma | 71 | — |
| | Endometrioid carcinoma | 9 | — |
| | Mucinous carcinoma | 3 | — |
| | Clear cell carcinoma | 5 | — |
| | Poorly differentiated carcinoma | 32 | — |
| | Metastatic carcinoma | 39 | — |
| | Serous border line tumours | 6 | — |
| | Others | 7 | — |
| Breast | Invasive ductal carcinoma | 451 | — |
| | Invasive lobular carcinoma | 49 | — |
| | Poorly differentiated carcinoma | 41 | — |
| | Medullary carcinoma | 9 | — |
| | Mucinous or papillary carcinoma | 18 | — |
| | Axillary node metastases | 31 | — |
| | Intraductal carcinoma | 41 | — |
| | Phyllodes tumor | 41 | — |
| | Others | 6 | — |
| Bone & soft tissue | Ewing tumor | 30 | — |
| | Rhabdomyosarcoma | 25 | — |
| | Desmoplastic tumor | 24 | — |
| | Neuroblastoma | 21 | — |
| | Fibromatosis | 4 | — |
| Others | | 4 | — |
| | | 1241 | |

### MCV350 DNA Sequences in Merkel Cell Carcinoma.

DNA extracted from frozen tumour tissue or cells was analysed by PCR for the presence of MCV350 DNA sequences using primers designed in the ST, LT, or VP1 sequences (cf supplementary data). All 10 cases of MCC were positive for MCV (table 3). DNA fragments with the expected sizes of 165, 162 and 204 bp were obtained in each case except in case N^{o} 1 for which only DNA corresponding to the ST and LT sequences could be amplified.

Q-PCR experiments using amplimers designed in the LT sequences of the MCV350 were performed to assess the number of viral genomes per cell in MCC. Viral DNA loads ranging from 0.6 to 10.3 genome-equivalent per carcinoma cell were observed in 9 cases. A much higher viral load of 62.2 was detected in one case (N^{o} 5) (table 3) which was further analysed using contiguous and inversely oriented primers. This experiment allowed the amplification of the whole viral genome (5387 bp), suggesting the presence of viral episomes. This genome was cloned. Sequencing showed that, in one of the clones isolated (MCV-IC13), ST, LT and VPs viral genes were fully conserved without mutation that could lead to truncated protein. This sequence showed a 99.3% identity rate with that of MCV350.

In order to verify that MCV 350 DNA sequences were located in the nucleus of epithelial tumour cells, we performed in situ hybridization analysis using the whole viral genome as a probe. FISH experiments were performed on frozen sections of case 8 which contains 6 copies of the MCV genome integrated at a single site. A single fluorescent signal was observed in the nucleus of epithelial tumour cells (data not shown). About 90% of the cells showed the signal, corresponding to the clonal pattern of integration. No significant signal was found in non tumour cells.

### MCV350 DNA Sequences in Non Merkel Cell Tumours

To determine whether MCV DNA sequences were present in tumour types other than MCC, detection of MCV350 sequences was performed by PCR using primers designed in the 5′ part of the LT sequences found to be conserved in all MCC cases. A total of 1241 specimens, taken from tumours of epithelial or mesenchymal origin, developed in adults or children, were included in the analysis (table 4). In none of these 1241 different specimens was there evidence of DNA likely to correspond to MCV sequences.

### MCV Genes Expression in Merkel Cell Carcinoma

Frozen tissue specimens from 8 of our 10 cases of MCC were available for RNA extraction. Total RNA was treated with DNAse to avoid the amplification of viral DNA. Quantitative RT-PCR was performed using amplimers designed in ST and LT sequences. Expression level of the TBP human gene was used as reference and three cervical cancer cell lines (IC1, 2, 3) negative for MCV were used as control. RNAs from the MCV sequences were expressed in all 8 cases. LT expression level ranged from 1.46 to 21.56 fold TBP and ST from 0.21 to 3.16 (table 3). No significant correlation between viral DNA load and viral RNA expression level was observed.

### Chromosome Localization of the Viral DNA Sequences.

Integration of viral DNA sequences into the tumour cell genome was investigated using the DIPS-PCR method which allows the localisation of the viral integration site at the molecular level. The integration site was identified in all 10 cases with primers designed to determine either the 3′ or the 5′ virus-host junctions. All cases harboured a single integration site which was found in 10 different loci (table 3). Viral DNA sequences were found inserted in the long arm of chromosomes 2, 3, 4, 5, 6, 8, 12, 20 and Y. In case N^{o} 6, the same chromosome localisation was observed in the primary and in the two skin metastases, demonstrating the clonality of the insertional mutation. In case N^{o} 8, the viral genome was interrupted at base 3119, at the junction between LT and VP1 sequences. Only 70 bp of cell DNA at the 5′ virus-host junction could be amplified. When compared to the human database, the specificity of this short sequence was not sufficient to correspond to a unique locus.

Analysis of the virus-host junctions allowed specification of the pattern of the integrated viral sequences. In 6 cases (N^{o} 1, 5, 6, 7, 9, 10), the virus-host junction was located in the 3′ part of the LT sequences (between nt 1515 and nt 2980) which were thus partly deleted (FIG. 1). In two cases (N^{o} 4, 2), the 3′ virus-host junction was located in VP1 (nt 3712) or in the regulatory region (nt 5202) and the LT sequences were fully conserved. LT DNA sequencing showed the presence of a 72 bp deletion (1403-1480) leading to a stop codon (PY261X) (case N^{o} 2) and the presence of a mutation (1390) leading to a stop codon (PQ255X) (case N^{o} 4). In two cases (N^{o} 3, 8), only the 5′ host-virus junction was identified and the localisation of the 3′ break point regarding LT sequences could not be specified. In all 8 informative cases, the 3′ part of integrated viral LT sequences was prematurely truncated (FIG. 1). In all 10 cases, ST sequences were fully conserved.

### Status of Cellular Genes Potentially Involved in Oncogenesis and Located at the Vicinity of the Integration Sites.

The possibility that integration of MCV DNA could lead to the deregulation of cellular genes involved in the tumour process was investigated. The genes located in the vicinity of the integrated viral sequences were identified. Viral sequences were found to be located in the AX747640 and SNAT1 genes (cases N^{o} 2 and N^{o} 3), at 1.3 kb from the IL20RA gene (case N^{o} 9), at 1 Mb from the SRD5A2L2 gene (case N^{o} 5) and at 1.35 Mb from MYC (case N^{o} 1) (table 3). Since MYC has been found activated by viral insertion in human tumours [21] and IL20 RA inactivation implicated in lung carcinogenesis [22] the expression level of these genes was further assessed by RT-PCR for 8 of the 10 cases. No significant gene deregulation related to MCV viral insertion was found (table 3).

### Array-CGH Analysis

Cellular DNA sequence copy number changes are reported in table 3. Three of the 10 samples analysed did not show any imbalance. Recurrent imbalances were gains of 1q (2 cases), 6p (3 cases), and 1l (2 cases), and loss of 17p (2 cases). No correlation was found between these chromosome rearrangements and integration sites of the MCV.

### REFERENCES

Throughout this application, various references describe the state of the art to which this invention pertains. The disclosures of these references are hereby incorporated by reference into the present disclosure.
1. Toker C. Trabecular carcinoma of the skin. Arch Dermatol 1972; 105:107-110
2. Sibley R K, Rosai J, Foucar E, Dehner L P and Bosl G. Neuroendocrine (Merkel cell) carcinoma of the skin. A histologic and ultrastructural study of two cases. Am J Surg Pathol 1980; 4:211-221
3. Frigerio B, Capella C, Eusebi V, Tenti P and Azzopardi J G. Merkel cell carcinoma of the skin: the structure and origin of normal Merkel cells. Histopathology 1983; 7:229-249
4. Llombart B, Monteagudo C, Lopez-Guerrero J A, Carda C, Jorda E, Sanmartin O, et al. Clinicopathological and immunohistochemical analysis of 20 cases of Merkel cell carcinoma in search of prognostic markers. Histopathology 2005; 46:622-634
5. Swann M H and Yoon J. Merkel cell carcinoma. Semin Oncol 2007; 34:51-56
6. Hodgson N C. Merkel cell carcinoma: changing incidence trends. J Surg Oncol 2005; 89:1-4
7. Lemos B and Nghiem P. Merkel cell carcinoma: more deaths but still no pathway to blame. J Invest Dermatol 2007; 127:2100-2103
8. Feng H, Shuda M, Chang Y and Moore P S. Clonal integration of a polyomavirus in human Merkel cell carcinoma. Science 2008; 319:1096-1100
9. Kassem A, Schopflin A, Diaz C, Weyers W, Stickeler E, Werner M, et al. Frequent detection of Merkel cell polyomavirus in human Merkel cell carcinomas and identification of a unique deletion in the VP1 gene. Cancer Res 2008; 68:5009-5013
10. Becker J C, Houben R, Ugurel S, Trefzer U, Pfohler C and Schrama D. MC polyomavirus is frequently present in Merkel cell carcinoma of European patients. J Invest Dermatol 2009; 129:248-250
11. Garneski K M, Warcola A H, Feng Q, Kiviat N B, Leonard J H and Nghiem P. Merkel cell polyomavirus is more frequently present in North American than Australian Merkel cell carcinoma tumors. J Invest Dermatol 2009; 129:246-248
12. Gardner S D, Field A M, Coleman D V and Hulme B. New human papovavirus (B.K.) isolated from urine after renal transplantation. Lancet 1971; 1:1253-1257
13. Padgett B L, Walker D L, ZuRhein G M, Eckroade R J and Dessel B H. Cultivation of papova-like virus from human brain with progressive multifocal leucoencephalopathy. Lancet 1971; 1:1257-1260
14. Allander T, Andreasson K, Gupta S, Bjerkner A, Bogdanovic G, Persson M A, et al. Identification of a third human polyomavirus. J Virol 2007; 81:4130-4136
15. Gaynor A M, Nissen M D, Whiley D M, Mackay I M, Lambert S B, Wu G, et al. Identification of a novel polyomavirus from patients with acute respiratory tract infections. PLoS Pathog 2007; 3:e64
16. zur Hausen H. Novel human polyomaviruses—re-emergence of a well known virus family as possible human carcinogens. Int J Cancer 2008; 123:247-250
17. Asioli S, Righi A, Volante M, Eusebi V and Bussolati G. p63 expression as a new prognostic marker in Merkel cell carcinoma. Cancer 2007; 110:640-647
18. Livak K J and Schmittgen T D. Analysis of relative gene expression data using real-time quantitative PCR and the 2(-Delta Delta C(T)) Method. Methods 2001; 25:402-408
19. Luft F, Klaes R, Nees M, Dürst M, Heilmann V, Melsheimer P, et al. Detection of integrated papillomavirus sequences by ligation-mediated PCR (DIPS-PCR) and molecular characterization in cervical cancer cells. Int J Cancer 2001; 92:9-17
20. La Rosa P, Viara E, Hupe P, Pierron G, Liva S, Neuvial P, et al. VAMP: visualization and analysis of array-CGH, transcriptome and other molecular profiles. Bioinformatics 2006; 22:2066-2073
21. Peter M, Rosty C, Couturier J, Radvanyi F, Teshima H and Sastre-Garau X. MYC activation associated with the integration of HPV DNA at the MYC locus in genital tumors. Oncogene 2006; 25:5985-5993
22. Tessema M, Willink R, Do K, Yu Y Y, Yu W, Machida E O, et al. Promoter methylation of genes in and around the candidate lung cancer susceptibility locus 6q23-25. Cancer Res 2008; 68:1707-1714.

1. A virus-like particle (VLP) wherein said VLP is a polyomavirus comprising a nucleic acid sequence having at least 60% identity with SEQ ID NO:5 (exon 2 of large T antigen, LT) and wherein said VLP has been isolated from a patient, preferably a patient suffering from Merkel Cell Carcinoma (MCC) in an episomal form or integrated in the patient's genome.

2. A VLP according to claim 1 comprising a polypeptide of at least 470 amino acids having at least 60% identity with SEQ ID NO:6 (LT) over said at least 470 amino acids.

3. A VLP according to claim 2 comprising a polypeptide having at least 60% identity with SEQ ID NO:6.

4. A VLP according to claim 1, further comprising at least one nucleic acid selected from the group consisting of the nucleic acids having:
at least 99.4% identity with SEQ ID NO: 5 (exon 2 of LT);
at least 99.2% identity with SEQ ID NO:4 (exon 1 of LT);
at least 99.5% identity with SEQ ID NO:2 (ST);
at least 99.5% identity with SEQ ID NO:7 (VP1);
at least 99.5% identity with SEQ ID NO:9 (VP2);
at least 99.5% identity with SEQ ID NO:11 (VP3) and
at least 99.4% identity with SEQ ID NO:1 (full genome of MCV-IC13).

5. An isolated nucleic acid selected from the group consisting of a nucleic acid having at least 99.4% identity with SEQ ID NO:1, a nucleic acid having at least 99.5% identity with SEQ ID NO:2, a nucleic acid having at least 99.2% identity with SEQ ID NO:4, a nucleic acid having at least 99.4% identity with SEQ ID NO:5, a nucleic acid having at least 99.5% identity with SEQ ID NO:7, a nucleic acid having at least 99.5% identity with SEQ ID NO:9 and a nucleic acid having at least 99.5% identity with SEQ ID NO:11.

6. A isolated polypeptide selected from the group consisting of an amino acid sequence having at least 99.6% identity with SEQ ID NO:3, an amino acid sequence having at least 98.6% identity with SEQ ID NO:6, an amino acid sequence having at least 99.4% identity with SEQ ID NO:8, an amino acid sequence having at least 99.3% identity with SEQ ID NO:10 and an amino acid sequence having at least 99.0% identity with SEQ ID NO:12, or a fragment of said polypeptide having at least 99.6% identity with the corresponding fragments of SEQ ID NO:3, SEQ ID NO:6, SEQ ID NO:8, SEQ ID NO:10 and SEQ ID NO:12, respectively.

7. An anti-MCV agent wherein said anti-MCV agent is a molecule which specifically interacts with a nucleic acid according to claim 5.

8. An anti-MCV agent wherein said anti-MCV agent inhibits the expression and/or the activity of at least one nucleic acid according to claim 5.

9. An antibody which specifically recognizes the non-truncated LT protein.

10. An antibody according to claim 9, wherein said antibody specifically recognizes an antigen comprised between amino acids 456 to 817 of SEQ ID NO: 6.

11. An antibody according to claim 9, wherein said antibody specifically recognizes a conformational epitope wherein said conformational epitope is partly comprised of residues located between amino acids 456 to 817 of SEQ ID NO: 6.

12. A method for detecting a VLP according to claim 1, comprising the step of detecting a nucleic acid selected from the group consisting of a nucleic acid having at least 99.4% identity with SEQ ID NO:1, a nucleic acid having at least 99.5% identity with SEQ ID NO:2, a nucleic acid having at least 99.2% identity with SEQ ID NO:4, a nucleic acid having at least 99.4% identity with SEQ ID NO:5, a nucleic acid having at least 99.5% identity with SEQ ID NO:7, a nucleic acid having at least 99.5% identity with SEQ ID NO:9 and a nucleic acid having at least 99.5% identity with SEQ ID NO:11; or detecting a polypeptide selected from the group consisting of an amino acid sequence having at least 99.6% identity with SEQ ID NO:3, an amino acid sequence having at least 98.6% identity with SEQ ID NO:6, an amino acid sequence having at least 99.4% identity with SEQ ID NO:8, an amino acid sequence having at least 99.3% identity with SEQ ID NO:10 and an amino acid sequence having at least 99.0% identity with SEQ ID NO:12, or a fragment of said polypeptide having at least 99.6% identity with the corresponding fragments of SEQ ID NO:3, SEQ ID NO:6, SEQ ID NO:8, SEQ ID NO:10 and SEQ ID NO:12, respectively.

13. A method according to claim 12, comprising the step of detecting an LT protein with an antibody which specifically recognizes the non-truncated LT protein.

14. A method for predicting the risk of developing an MCV-associated disease in a patient comprising the step of detecting a VLP according to claim 1 in a tissue sample obtained from said patient.

15. A method for diagnosing an MCV-associated disease in a patient comprising the step of detecting a VLP according to claim 1 in a tissue sample obtained from said patient.

16. A method according to claim 14, wherein said MCV-associated disease is Merkel Cell Carcinoma (MCC).

17. A kit for diagnosis an MCV-associated disease in a patient comprising an anti-MCV agent according to claim 7 and means for revealing said anti-MCV agent or antibody.

18. A method for identifying an agent that attenuates MCV infection comprising the step of exposing a target DNA to a polypeptide according to claim 6 in the presence or absence of a test compound.

19. A pharmaceutical composition comprising:
a VLP according to and/or
a nucleic acid selected from the group consisting of a nucleic acid having at least 99.4% identity with SEQ ID NO:1, a nucleic acid having at least 99.5% identity with SEQ ID NO:2, a nucleic acid having at least 99.2% identity with SEQ ID NO:4, a nucleic acid having at least 99.4% identity with SEQ ID NO:5, a nucleic acid having at least 99.5% identity with SEQ ID NO:7, a nucleic acid having at least 99.5% identity with SEQ ID NO:9 and a nucleic acid having at least 99.5% identity with SEQ ID NO:11 and/or
a polypeptide selected from the group consisting of an amino acid sequence having at least 99.6% identity with SEQ ID NO:3, an amino acid sequence having at least 98.6% identity with SEQ ID NO:6, an amino acid sequence having at least 99.4% identity with SEQ ID NO:8, an amino acid sequence having at least 99.3% identity with SEQ ID NO:10 and an amino acid sequence having at least 99.0% identity with SEQ ID NO:12, or a fragment of said polypeptide having at least 99.6% identity with the corresponding fragments of SEQ ID NO:3, SEQ ID NO:6, SEQ ID NO:8, SEQ ID NO:10 and SEQ ID NO:12, respectively and/or
an anti-MCV agent which is a molecule which specifically interacts with a nucleic acid selected from the group consisting of a nucleic acid having at least 99.4% identity with SEQ ID NO:1, a nucleic acid having at least 99.5% identity with SEQ ID NO:2, a nucleic acid having at least 99.2% identity with SEQ ID NO:4, a nucleic acid having at least 99.4% identity with SEQ ID NO:5, a nucleic acid having at least 99.5% identity with SEQ ID NO:7, a nucleic acid having at least 99.5% identity with SEQ ID NO:9 and a nucleic acid having at least 99.5% identity with SEQ ID NO:11, or specifically interacts with a polypeptide selected from the group consisting of an amino acid sequence having at least 99.6% identity with SEQ ID NO:3, an amino acid sequence having at least 98.6% identity with SEQ ID NO:6, an amino acid sequence having at least 99.4% identity with SEQ ID NO:8, an amino acid sequence having at least 99.3% identity with SEQ ID NO:10 and an amino acid sequence having at least 99.0% identity with SEQ ID NO:12, or a fragment of said polypeptide having at least 99.6% identity with the corresponding fragments of SEQ ID NO:3, SEQ ID NO:6, SEQ ID NO:8, SEQ ID NO:10 and SEQ ID NO:12, respectively and a pharmaceutically acceptable carrier.
